(19) United States
(12) Patent Application Publication
(10) Pub. No.: US 2025/0118223 A1
(43) Pub. Date: Apr. 10, 2025

(54) AUTOMATIC DETECTION SYSTEM AND DEVICE FOR CAPTURING ICTAL SEMIOLOGY OF EPILEPTIC PATIENT

(71) Applicant: XUANWU HOSPITAL, CAPITAL MEDICAL UNIVERSITY, Beijing (CN)

(72) Inventor: Yicong LIN, Beijing (CN)

(21) Appl. No.: 18/850,666

(22) PCT Filed: Jul. 22, 2024
(86) PCT No.: PCT/CN2024/106623

§ 371 Date: Sep. 25, 2024

(30) Foreign Application Priority Data
Sep. 25, 2023 (CN) 202311241047.2

(51) Int. Cl. G09B 23/28; A61B 5/00; A61B 5/38

(52) U.S. Cl. CPC G09B 23/28; A61B 5/38; A61B 5/4094

(57) ABSTRACT

This application provides an automatic detection system and device for capturing ictal semiology of an epileptic patient, and relates to the field of biomedical detection. When an epileptic patient undergoes a premonitory seizure, a data processing module or an electroencephalogram marking machine triggers a voice module to play a voice command. When an epileptic patient undergoes a premonitory seizure, the voice command instructs the epileptic patient to describe a subjective feeling and instructs the epileptic patient to complete examination of a consciousness level and a motor ability during an ictal phase. Through the voice module, an audio of the subjective feeling of the epileptic patient is automatically recorded synchronously in electroencephalogram monitoring data. Through a camera, a video of examination results of the consciousness level and the motor ability of the epileptic patient during the ictal phase is automatically recorded synchronously in the electroencephalogram monitoring data.

### CROSS REFERENCE TO RELATED APPLICATION

The present application is a national stage application of International Patent Application No. PCT/CN2024/106623, filed on Jul. 22, 2024, which claims priority to Chinese Patent Application No. 202311241047.2 filed to the China National Intellectual Property Administration (CNIPA) on Sep. 25, 2023 and entitled “AUTOMATIC DETECTION SYSTEM FOR CAPTURING ICTAL SEMIOLOGY OF EPILEPTIC PATIENT”, which is incorporated herein by reference in its entirety.

### TECHNICAL FIELD

The present application belongs to the technical field of biomedical detection, and specifically relates to an automatic detection system and device for capturing ictal semiology of an epileptic patient.

### BACKGROUND

Premonitory seizures in epileptic patients refer to subjective abnormal feelings at the very beginning of an epileptic seizure in the epileptic patients, such as visual auras (flashes in front of eyes), auditory auras (hearing sounds or words), and sensory auras (abdominal discomfort, smelling peculiar smells, having fevers and chills, and dizziness). The timely and accurate capture and the synchronous recording with an electroencephalogram of these symptoms is very important for locating a brain region in a patient where an epileptic seizure starts and for classifying an epileptic seizure. The current method for accurately recording premonitory seizures in patients whose electroencephalograms are monitored is as follows: during the monitoring of an electroencephalogram, a patient is instructed to actively press a marking machine when a premonitory seizure occurs (a marker symbol for a corresponding time point will appear on the electroencephalogram synchronously recorded). However, this method cannot synchronously record the subjective feelings of a patient into the electroencephalogram data. As a result: 1) A patient cannot accurately recall the information of premonitory symptoms afterwards. 2) The premonitory information inquired by a doctor later is physically separated from the electroencephalogram data, such that, during data rereading analysis in the future, only the electroencephalogram data can be seen and the premonitory information loses.

The ictal phase is very important for the examination of a consciousness level and a motor ability of a patient, and can help determine an impact of an epileptic discharge in the ictal phase on a brain region and side. However, currently, when electroencephalogram monitoring is conducted for an epileptic patient to capture symptom information during the ictal phase, the medical staff usually cannot rush to a bedside of a patient for neurological examination (namely, the examination of a consciousness level and a motor ability of the patient) in time during the ictal phase, such that the assessment information of the consciousness level and motor ability during the ictal phase are omitted, which reduces the clinical significance of electroencephalogram monitoring.

In view of the above problems, the medical staff can currently be trained regularly. That is, the medical staff are instructed to rush to a bedside of a patient in time for command actions and physical examination (namely, the examination of a consciousness level and a motor ability of the patient) when the patient undergoes an epileptic seizure, inquire premonitory symptoms of the patient after the epileptic seizure, and record the premonitory symptoms in a medical record (which are manually recorded in the medical record by the medical staff rather than directly recorded in electroencephalogram data synchronously). Therefore, how to automatically record subjective feelings of an epileptic patient undergoing a premonitory seizure synchronously in electroencephalogram monitoring data of the epileptic patient and automatically record examination results of a consciousness level and a motor ability of the epileptic patient during the ictal phase synchronously has become an urgent problem to be solved by those skilled in the art.

### SUMMARY

In view of the above technical problems, the present application provides an automatic detection system and device for capturing ictal semiology of an epileptic patient, which can automatically record subjective feelings of an epileptic patient undergoing a premonitory seizure synchronously in electroencephalogram monitoring data of the epileptic patient and automatically record examination results of a consciousness level and a motor ability of the epileptic patient during an ictal phase synchronously.

To allow the above objective, the present application provides the following solutions:
An automatic detection system for capturing ictal semiology of an epileptic patient is provided, including a data processing module and a voice module, where a voice command is stored in the voice module;
the data processing module is connected to the voice module; the data processing module is configured to acquire an electroencephalogram signal in a video electroencephalogram device in real time, process the electroencephalogram signal to obtain a power, and send a trigger signal to the voice module when the power exceeds a set threshold; when the power exceeds the set threshold, it is indicated that the epileptic patient begins to undergo a premonitory seizure; the trigger signal is configured to trigger a play of the voice command in the voice module;
the video electroencephalogram device includes an electroencephalogram marking machine; when the epileptic patient undergoes the premonitory seizure, the electroencephalogram marking machine is actively pressed, and a switch mechanical signal of the electroencephalogram marking machine is sent to the voice module; the switch mechanical signal is configured to trigger the play of the voice command in the voice module;
the voice module is connected to the video electroencephalogram device; the voice module is configured to trigger the play of the voice command in the voice module immediately after receiving the trigger signal sent by the data processing module or the switch mechanical signal sent by the electroencephalogram marking machine; the voice command in the voice module is configured to: when the epileptic patient undergoes the premonitory seizure, instruct the epileptic patient to describe a subjective feeling and instruct the epileptic patient to complete examination of a consciousness level and a motor ability during an ictal phase; the voice module is further configured to automatically record an audio of the subjective feeling of the epileptic patient synchronously in electroencephalogram monitoring data when the epileptic patient describes the subjective feeling; the video electroencephalogram device further includes a camera; and the camera is configured to: while the voice command in the voice module instructs the epileptic patient to complete the examination of the consciousness level and the motor ability of the epileptic patient during the ictal phase, capture examination results of the consciousness level and the motor ability of the epileptic patient during the ictal phase and automatically record a video of the examination results of the consciousness level and the motor ability of the epileptic patient during the ictal phase synchronously in the electroencephalogram monitoring data.

Optionally, the processing of the electroencephalogram signal by the data processing module includes fast fourier transform (FFT) and power spectrum analysis of a gamma frequency band.

Optionally, after the voice command in the voice module is triggered to be played, the voice command in the voice module is played circularly.

Optionally, the automatic detection system further includes a stop button; the stop button is connected to the voice module; when the stop button is pressed, a play-stop signal is sent to the voice module; and the play-stop signal is configured to control the voice module to stop the play of the voice command.

Optionally, the automatic detection system is embedded into the video electroencephalogram device.

Optionally, the voice command is a pre-recorded audio.

Optionally, the pre-recorded audio includes: “say how you feel at the moment”, “remember a red color”, “repeat after me: sun”, “close your eyes”, “open your eyes”, “raise your right upper limb”, “lower your right upper limb”, and “raise both your upper limbs above your head”.

An automatic detection apparatus for capturing ictal semiology of an epileptic patient is provided, including: a video electroencephalogram device and the automatic detection system for capturing ictal semiology of an epileptic patient described above, where the automatic detection system is embedded into the video electroencephalogram device; the video electroencephalogram device includes: an electroencephalogram marking machine and a camera; when the epileptic patient undergoes a premonitory seizure, the electroencephalogram marking machine is actively pressed, the electroencephalogram marking machine generates a switch mechanical signal, and the switch mechanical signal is sent to a voice module in the automatic detection system; the voice module is configured to trigger a play of a voice command immediately after receiving a trigger signal sent by a data processing module in the automatic detection system or the switch mechanical signal sent by the electroencephalogram marking machine; the voice command in the voice module is configured to: when the epileptic patient undergoes the premonitory seizure, instruct the epileptic patient to describe a subjective feeling and automatically record an audio of the subjective feeling of the epileptic patient synchronously in electroencephalogram monitoring data; the voice module is further configured to instruct the epileptic patient to make actions reflecting a consciousness level and a motor ability during an ictal phase; and the camera is configured to: while the voice command in the voice module instructs the epileptic patient to make the actions reflecting the consciousness level and the motor ability, capture the actions of the epileptic patient and automatically record a captured video synchronously in the electroencephalogram monitoring data.

The present application has the following beneficial effects:
With the automatic detection system and device for capturing ictal semiology of an epileptic patient disclosed in the present application: When the epileptic patient undergoes a premonitory seizure, the data processing module or the electroencephalogram marking machine triggers the voice module to play the voice command. When the epileptic patient undergoes a premonitory seizure, the voice command instructs the epileptic patient to describe a subjective feeling and instructs the epileptic patient to complete examination of a consciousness level and a motor ability during an ictal phase. Through the voice module, when the epileptic patient describes a subjective feeling, an audio of the subjective feeling of the epileptic patient is automatically recorded synchronously in electroencephalogram monitoring data. Through the camera of the video electroencephalogram device, while the voice command instructs the epileptic patient to complete examination of a consciousness level and a motor ability during an ictal phase, examination results of the consciousness level and the motor ability of the epileptic patient during the ictal phase are captured and a video of the examination results of the consciousness level and the motor ability of the epileptic patient during the ictal phase is automatically recorded synchronously in electroencephalogram monitoring data. In this way, the present application allows the automatic recording of subjective feelings of an epileptic patient undergoing a premonitory seizure synchronously in electroencephalogram monitoring data of the epileptic patient and the automatic recording of examination results of a consciousness level and a motor ability of the epileptic patient during an ictal phase synchronously.

### BRIEF DESCRIPTION OF THE DRAWINGS

To describe the technical solutions in the embodiments of the present application or in the prior art clearly, the accompanying drawings required for the embodiments are briefly described below. Apparently, the accompanying drawings in the following description show merely some embodiments of the present application, and those of ordinary skill in the art may still derive other accompanying drawings from these accompanying drawings without creative efforts.

FIG. 1 is a schematic diagram for a structure of an automatic detection system for capturing ictal semiology of an epileptic patient in an embodiment of the present application;

FIG. 2 is a connection diagram for a basic structure of an automatic detection system for capturing ictal semiology of an epileptic patient in an embodiment of the present application; and

FIG. 3 is a working diagram for an automatic detection system for capturing ictal semiology of an epileptic patient in an embodiment of the present application.

### DETAILED DESCRIPTION OF THE EMBODIMENTS

The technical solutions in the embodiments of the present application are clearly and completely described below with reference to the accompanying drawings in the embodiments of the present application. Apparently, the described embodiments are merely some rather than all of the embodiments of the present application. All other embodiments obtained by a person of ordinary skill in the art based on the embodiments of the present disclosure without creative efforts shall fall within the protection scope of the present disclosure.

An objective of the present application is to provide an automatic detection system for capturing ictal semiology of an epileptic patient, which can automatically record subjective feelings of an epileptic patient undergoing a premonitory seizure synchronously in electroencephalogram monitoring data of the epileptic patient and automatically record examination results of a consciousness level and a motor ability of the epileptic patient during an ictal phase synchronously.

In order to make the objective, features, and advantages of the present application clear and comprehensible, the present application will be further described in detail below in combination with accompanying drawings and specific implementations.

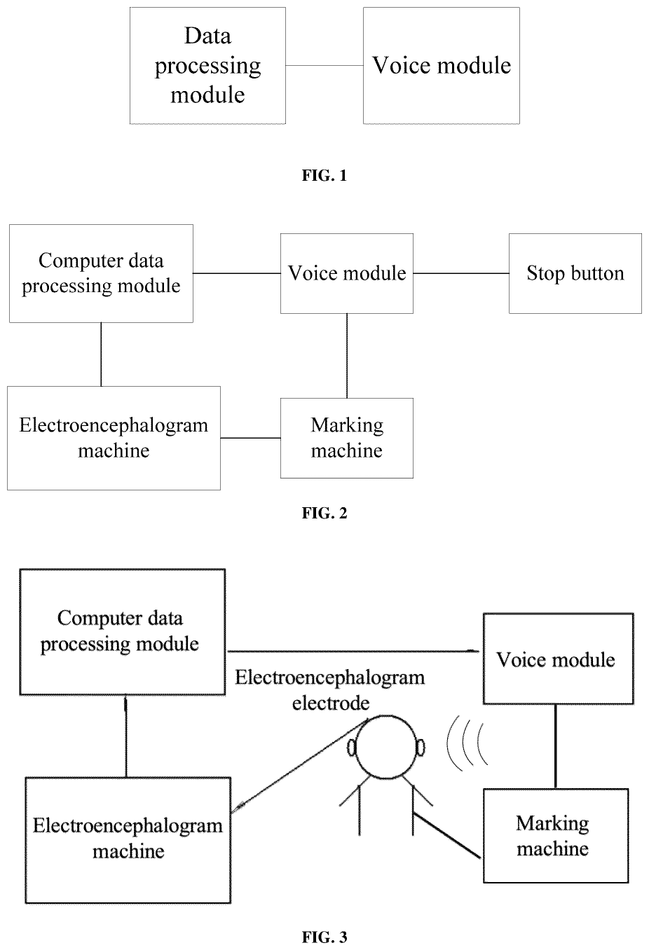

FIG. 1 is a schematic diagram for a structure of the automatic detection system for capturing ictal semiology of an epileptic patient in the embodiment of the present application. As shown in FIG. 1, the automatic detection system for capturing ictal semiology of an epileptic patient provided in the embodiment of the present application includes a data processing module and a voice module. A voice command is stored in the voice module.

The data processing module is connected to the voice module. The data processing module is configured to acquire an electroencephalogram signal in a video electroencephalogram device in real time, process the electroencephalogram signal to obtain a power, and send a trigger signal to the voice module when the power exceeds a set threshold. When the power exceeds the set threshold, it is indicated that the epileptic patient begins to undergo a premonitory seizure. The trigger signal is configured to trigger a play of the voice command in the voice module. The processing of the electroencephalogram signal by the data processing module includes FFT and power spectrum analysis of a gamma frequency band.

The video electroencephalogram device includes an electroencephalogram marking machine. When the epileptic patient undergoes the premonitory seizure, the electroencephalogram marking machine is actively pressed, and a switch mechanical signal of the electroencephalogram marking machine is sent to the voice module. The switch mechanical signal is configured to trigger the play of the voice command in the voice module.

The voice module is connected to the video electroencephalogram device. The voice module is configured to trigger the play of the voice command in the voice module immediately after receiving the trigger signal sent by the data processing module or the switch mechanical signal sent by the electroencephalogram marking machine. The voice command in the voice module is configured to: when the epileptic patient undergoes the premonitory seizure, instruct the epileptic patient to describe a subjective feeling and instruct the epileptic patient to complete examination of a consciousness level and a motor ability during an ictal phase. The voice module is further configured to automatically record an audio of the subjective feeling of the epileptic patient synchronously in electroencephalogram monitoring data when the epileptic patient describes the subjective feeling. The video electroencephalogram device further includes a camera. The camera is configured to: while the voice command in the voice module instructs the epileptic patient to complete the examination of the consciousness level and the motor ability of the epileptic patient during the ictal phase, capture examination results of the consciousness level and the motor ability of the epileptic patient during the ictal phase and automatically record a video of the examination results of the consciousness level and the motor ability of the epileptic patient during the ictal phase synchronously in the electroencephalogram monitoring data.

Optionally, the automatic detection system for capturing ictal semiology of an epileptic patient further includes a stop button. The stop button is connected to the voice module. When the stop button is pressed, a play-stop signal is sent to the voice module. The play-stop signal is configured to control the voice module to stop the play of the voice command. After the voice command in the voice module is triggered to be played, the voice command in the voice module is played circularly. When the voice module receives the play-stop signal, the play of the voice command is stopped.

The voice command is a pre-recorded audio. The pre-recorded audio includes: “say how you feel at the moment”, “remember a red color”, “repeat after me: sun”, “close your eyes”, “open your eyes”, “raise your right upper limb”, “lower your right upper limb”, and “raise both your upper limbs above your head”.

The technical solution of the present application is illustrated below through a specific embodiment;

FIG. 2 is a connection diagram for a basic structure of the automatic detection system for capturing ictal semiology of an epileptic patient in the embodiment of the present application. FIG. 3 is a working diagram for the automatic detection system for capturing ictal semiology of an epileptic patient in the embodiment of the present application. As shown in FIG. 2 and FIG. 3, the automatic detection system (automatic monitoring system) for capturing ictal semiology of an epileptic patient in the embodiment of the present application includes a voice module allowing pre-recording and a data processing module.

(1) The switch mechanical signal of the electroencephalogram marking machine (namely, a marking machine, which is a standard configuration of the existing video electroencephalogram device) is associated with the voice module. After the epileptic patient marks manually, the switch mechanical signal is transmitted to a pre-recorded voice module, such that a pre-recorded voice command in the voice module can be triggered and played circularly until the stop button (stop key) is manually pressed. The marking of the epileptic patient is intended to trigger a recorded voice content in the voice module. The pressing of the stop button needs to be conducted by the external medical staff rather than the epileptic patient.

Specifically, after a voice play is triggered, the voice command instructs the epileptic patient to:
1. “say how you feel at the moment”;
2. “remember a red color”;
3. “repeat after me: sun”;
4. “close your eyes” and “open your eyes”;
5. “raise your right upper limb” and “lower your right upper limb”; and
6. “raise both your upper limbs above your head”.

The voice command can be replayed once.

The existing video electroencephalogram device is provided with a camera. While the voice module instructs the epileptic patient to conduct the above-mentioned series of actions, the camera of the video electroencephalogram device captures the actions of the epileptic patient to provide a data support for medical staff.

(2) The data processing module (computer data processing module) is configured to continuously acquire an electroencephalogram signal in the existing video electroencephalogram device (video electroencephalogram instrument) in real time. The data processing module adopts FFT and power spectrum analysis of a gamma frequency band. When a power exceeds a set threshold, the pre-recorded voice command in the voice module can be triggered and played circularly until the stop button is manually pressed. The electroencephalogram signal can be acquired routinely by the existing video electroencephalogram device, and the existing device (which is provided with a camera, an electroencephalogram amplifier, a built-in spectrum analysis function for each frequency band, or the like). The Fourier transform and power spectrum analysis is a built-in function of the video electroencephalogram device. The acquisition and analysis of an electroencephalogram signal of the data processing module is intended to trigger the voice command in the voice module.

While the voice module instructs the epileptic patient to conduct the above-mentioned series of actions, the camera of the video electroencephalogram device captures the actions of the epileptic patient to provide a data support for medical staff.

A function of the voice module is to individually pre-record a command of a specified dialect for an ictal phase.

A function of the data processing module is to conduct real-time FFT and power spectrum analysis of a gamma frequency band for an electroencephalogram signal acquired by the electroencephalogram device (electroencephalogram machine) and send a signal to the voice module according to a set threshold for triggering. As shown in FIG. 3, electroencephalogram electrodes are attached to a surface of a scalp of the epileptic patient, and the electroencephalogram machine acquires the electroencephalogram signal through the electroencephalogram electrodes. The electroencephalogram machine is a structure in the video electroencephalogram device.

A voice prompt function of the voice module can be triggered through any one of the following two pathways (a doctor can determine which pathway to take according to needs before activating the function):

### 1. Triggering Through the Manual Marking of the Epileptic Patient:

After the electroencephalogram marking machine is pressed by the epileptic patient for marking, the voice play is automatically triggered immediately, which is a first pathway.

### 2. Triggering by the Data Processing Module:

After the built-in functional spectrum analysis of the video electroencephalogram device indicates that the energy increases suddenly (indicating that a seizure is about to begin) and exceeds a set threshold (which is set by a doctor individually for epileptic patients), the voice play is automatically triggered immediately, which is a second pathway.

An embodiment of the present application also provides an automatic detection apparatus for capturing ictal semiology of an epileptic patient, including: a video electroencephalogram device and the automatic detection system for capturing ictal semiology of an epileptic patient described above.

The automatic detection system is embedded into the video electroencephalogram device. The video electroencephalogram device includes: an electroencephalogram marking machine and a camera. When the epileptic patient undergoes a premonitory seizure, the electroencephalogram marking machine is actively pressed, the electroencephalogram marking machine generates a switch mechanical signal, and the switch mechanical signal is sent to a voice module in the automatic detection system.

The voice module is configured to trigger a play of a voice command immediately after receiving a trigger signal sent by a data processing module in the automatic detection system or the switch mechanical signal sent by the electroencephalogram marking machine. The voice command in the voice module is configured to: when the epileptic patient undergoes the premonitory seizure, instruct the epileptic patient to describe a subjective feeling and automatically record an audio of the subjective feeling of the epileptic patient synchronously in electroencephalogram monitoring data.

The voice module is further configured to instruct the epileptic patient to make actions reflecting a consciousness level and a motor ability during an ictal phase. The camera is configured to: while the voice command in the voice module instructs the epileptic patient to make the actions reflecting the consciousness level and the motor ability, capture the actions of the epileptic patient and automatically record a captured video synchronously in the electroencephalogram monitoring data.

Compared with the prior art, the present application has the following advantages:
1. An epileptic patient can mark to automatically trigger voice prompt information. When an epileptic patient marks, the voice prompt information (which is recorded in advance according to a dialect of the epileptic patient) is automatically triggered, which can instruct the epileptic patient to describe symptoms of subjective feelings and instruct the epileptic patient to complete a question-answer session and corresponding actions (which are recorded in advance according to the dialect of the epileptic patient). The examination includes instructing an epileptic patient to remember a color, point to a ceiling, repeat a sentence, raise a specific limb, or the like.
2. An audio and a video are recorded synchronously in electroencephalogram monitoring data.

Compared with the prior art, the present application has the following beneficial effects:
1. The automatic detection system for capturing ictal semiology of an epileptic patient in the present application can automatically prompt a voice command at the first time when the epileptic patient undergoes a premonitory seizure instead of the manual instruction of the epileptic patient to complete the examination, so as to capture the information of an early seizure (the symptom information of the early seizure is very important for the location of an origin of an epileptic seizure). In addition, the automatic detection system for capturing ictal semiology of an epileptic patient in the present application can synchronize auras recorded in a medical record with electroencephalogram monitoring data, which allows the direct corresponding to electroencephalogram signal evolution and seizure symptomatology characteristics, and allows the long-term trace retention without involving an information lose and affecting the retrospective analysis.
2. The automatic detection system for capturing ictal semiology of an epileptic patient in the present application is embedded into a video electroencephalogram device, which can increase the captured epileptic seizure information (namely, subjective feelings of the epileptic patient undergoing a premonitory seizure and examination results of a consciousness level and a motor ability of the epileptic patient during an ictal phase) and is conducive to the clinical diagnosis and treatment for epileptic patients.
3. The automatic detection system for capturing ictal semiology of an epileptic patient in the present application can automatically record a time point of a premonitory seizure in time while recording an electroencephalogram, conduct a question-answer session with the epileptic patient through an automatic voice control system, prompt the epileptic patient to say his/her abnormal feelings immediately after a seizure occurs, and record the abnormal feelings in the form of an audio synchronously in an electroencephalogram data system, which allows long-term trace retention.
4. The automatic detection system for capturing ictal semiology of an epileptic patient in the present application can automatically start a physical examination command for the epileptic patient immediately when the epileptic patient undergoes a premonitory seizure and marks to complete the evaluation of a consciousness level and a motor ability of the epileptic patient, and record evaluation results in the form of a video synchronously in an electroencephalogram data system, which allows long-term trace retention.

The embodiments of the present application have been described above with reference to the accompanying drawings, but the present application is not limited to the above specific implementations. The above specific implementations are merely illustrative rather than restrictive. Under the inspiration of the present application, those of ordinary skill in the art can make many variations without departing from the purpose of the present application and the protection scope defined by the claims, and these variations shall fall within the protection scope of the present disclosure.

1. An automatic detection system for capturing ictal semiology of an epileptic patient, comprising a data processing module and a voice module, wherein the automatic detection system is embedded into a video electroencephalogram device, a voice command is stored in the voice module; the voice command is a pre-recorded audio; and the pre-recorded audio comprises: “say how you feel at the moment”, “remember a red color”, “repeat after me: sun”, “close your eyes”, “open your eyes”, “raise your right upper limb”, “lower your right upper limb”, and “raise both your upper limbs above your head”;
the data processing module is connected to the voice module; the data processing module is configured to acquire an electroencephalogram signal in the video electroencephalogram device in real time, process the electroencephalogram signal to obtain a power, and send a trigger signal to the voice module when the power exceeds a set threshold; when the power exceeds the set threshold, it is indicated that the epileptic patient begins to undergo a premonitory seizure; the trigger signal is configured to trigger a play of the voice command in the voice module, wherein acquisition and analysis of the electroencephalogram signal by the data processing module aims to trigger the voice command in the voice module;
the video electroencephalogram device comprises an electroencephalogram marking machine; when the epileptic patient undergoes the premonitory seizure, the electroencephalogram marking machine is actively pressed, and a switch mechanical signal of the electroencephalogram marking machine is sent to the voice module; the switch mechanical signal is configured to trigger the play of the voice command in the voice module, wherein that the electroencephalogram marking machine is actively pressed aims to trigger a recorded voice content in the voice module;
the voice module is connected to the video electroencephalogram device; the voice module is configured to trigger the play of the voice command in the voice module immediately after receiving the trigger signal sent by the data processing module or the switch mechanical signal sent by the electroencephalogram marking machine; after the voice command in the voice module is triggered to be played, the voice command in the voice module is played circularly; the voice command in the voice module is configured to: when the epileptic patient undergoes the premonitory seizure, instruct the epileptic patient to describe a subjective feeling and instruct the epileptic patient to complete examination of a consciousness level and a motor ability during an ictal phase; the voice module is further configured to automatically record an audio of the subjective feeling of the epileptic patient synchronously in electroencephalogram monitoring data when the epileptic patient describes the subjective feeling; the video electroencephalogram device further comprises a camera; and the camera is configured to: while the voice command in the voice module instructs the epileptic patient to complete the examination of the consciousness level and the motor ability of the epileptic patient during the ictal phase, capture examination results of the consciousness level and the motor ability of the epileptic patient during the ictal phase and automatically record a video of the examination results of the consciousness level and the motor ability of the epileptic patient during the ictal phase synchronously in the electroencephalogram monitoring data.

2. The automatic detection system for capturing ictal semiology of an epileptic patient according to claim 1, wherein the processing of the electroencephalogram signal by the data processing module comprises fast fourier transform (FFT).

3. The automatic detection system for capturing ictal semiology of an epileptic patient according to claim 2, wherein the processing of the electroencephalogram signal by the data processing module further comprises power spectrum analysis of a gamma frequency band.

4. The automatic detection system for capturing ictal semiology of an epileptic patient according to claim 1, further comprising a stop button, wherein
the stop button is connected to the voice module; when the stop button is pressed, a play-stop signal is sent to the voice module; and the play-stop signal is configured to control the voice module to stop the play of the voice command.

5-8. (canceled)
